(19) United States
(12) Patent Application Publication
(10) Pub. No.: US 2020/0254158 A1
(43) Pub. Date: Aug. 13, 2020

(54) MINIMALLY INVASIVE SURGICAL DRAIN IMMOBILIZATION SYSTEM AND METHOD

(71) Applicants: Joshua Burkin, Daytona Beach, FL (US); Justin Bailey, Oviedo, FL (US)

(72) Inventors: Joshua Burkin, Daytona Beach, FL (US); Justin Bailey, Oviedo, FL (US)

(21) Appl. No.: 16/590,346

(22) Filed: Oct. 1, 2019

Related U.S. Application Data
(60) Provisional application No. 62/803,793, filed on Feb. 11, 2019.

(51) Int. Cl. A61M 1/00

(52) U.S. Cl. CPC A61M 1/0086; A61M 1/0088; A61M 1/0082

(57) ABSTRACT

A drain immobilization device for the securement or fixation of surgical drains during surgery or post-surgery to remove fluids accumulated in a wound site or other area, wherein instead of having the surgical tubes fastened to the patient's body by sutures or stitches the surgical tubes are held inside of a clasp, the clasp attached to a base, the base having a layer of adhesive to attach to the patient's skin.

### CROSS REFERENCE To RELATED APPLICATIONS

This application claims priority to a prior-filed provisional application Ser. No. 62/803,793 filed on Feb. 11, 2019.

### FIELD OF THE DISCLOSURE

This disclosure relates to the field of medical drainage devices. More specifically the field of the invention is devices and methods for the securement or fixation of surgical drains during use.

### BACKGROUND

Over the years, millions of surgical drains have been placed in patients worldwide. A surgical drain allows pus, blood or other fluids from a wound or other area to flow out of the patient's body. To accomplish this, a doctor will position a thin rubber tube into the area of the body where fluid is likely to collect. The rubber tube will then carry the fluid outside the body through a small incision wherein a squeeze bulb or other apparatus is attached to the rubber tube. The surgical drain uses suction created by the bulb to pull the fluid from the body into the bulb. The current standard of care, regarding surgical drains, has been the utilization of sutures or stitches wherein the rubber tube is held in place by one or two stitches to the patient's skin. The bulb is also attached with a safety pin to clothing or near the bandage so that it doesn't rotate or pull on the stitches.

This method is archaic in that a suture removal kit is used which normally requires a doctor's assistance because of the difficulty in removing the stitches by the patient themselves. The amount of time and money used in applying and then removing the stitches is not efficient as it increases the time in the operating room and the patient must return to the doctor to remove the stitches. Patients also prefer not to have additional procedures done on their body if it is not needed for not only safety concerns but cosmetic anxieties as well. Thus, exists a need for an innovation that is utilized in substitute of surgical drain stitches while providing the same practical benefits

### SUMMARY

The present invention is directed to a drain immobilization device, comprising, a clasp configured to secure a surgical drainage tube, the clasp connected to a base by an adhesive, the base configured to attach to a patient's skin, wherein adhesive is coated on at least a portion of a surface of the base, the base comprised of a vinyl material, the clasp comprised of a first component and second component, the first component pivotably rotatable to the second component, the first component and the second component each having a recess, the recess of the first and the second component integrally forming an aperture when the first component and the second component are parallel to one another, the aperture configured to receive the surgical drainage tube, the recess of the first and the second component having one or more studs along the length of the recess of the first and the second component, an actuator configured to lock and unlock the clasp, the actuator having a bottom end configured to be received into a recess in the sidewall of the bottom component, the actuator having a top end configured to be moved toward a sidewall of the top component such that when top end moves toward the sidewall of the top component the bottom end is moved away from the recess in the sidewall of the bottom component, the top end and the bottom end in the shape of a flexing curve, the top end facing outward from the sidewall of the top component and the bottom end facing inward toward the sidewall of the top component, a part of the actuator configured to connect to a recess in the sidewall of the top component, the connection configured to act as a pivot point for the actuator to rotate around.

The present invention is also directed to a drain immobilization device, comprising, a clasp configured to secure a surgical drainage tube, the clasp comprised of a first component and second component, the first component pivotably rotatable to the second component, the first component and the second component each having a recess, the recess of the first and the second component integrally forming an aperture when the first component and the second component are parallel to one another, the aperture configured to receive the surgical drainage tube, an actuator configured to lock and unlock the clasp, the actuator connected to the clasp such that the connection acts as a pivot point for the actuator, the actuator having a second end configured to be received into a securing recess in the clasp wherein when the second end is received into the securing recess the surgical drainage tube is secured in the aperture, the actuator having a first end configured to be moved toward the clasp such that when first end moves toward the clasp the second end is moved away from the securing recess in the clasp, the first end and the second end of the actuator are in the shape of a flexing curve, the first end facing outward from the sidewall of the first component and the second end facing inward toward the sidewall of the first component, the clasp connected to a base made of a vinyl material, the base configured to be attached to a patient's skin, wherein adhesive is coated on at least a portion of a surface of the base.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1 shows a perspective view of the surgical drain immobilization system in the closed position.

FIG. 2 shows a front view of the clasp of the surgical drain immobilization system in the closed position.

FIG. 3 shows a perspective view of the surgical drain immobilization system in the open position.

FIG. 4 shows a perspective view of the surgical drain immobilization system in use on a patient's body.

FIG. 5 shows a top perspective view of a second embodiment of the surgical drain immobilization system in the closed position.

FIG. 6 shows a top perspective view of the clasp of the surgical drain immobilization system in the open position.

FIG. 7 shows a front perspective view of the clasp of the surgical drain immobilization system in the open position.

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Certain terminology and derivations thereof may be used in the following description for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The present disclosure recognizes the unsolved need for an improved system and method for the securement of surgical drains during use, thus replacing surgical sutures that are fastened to the patient's body. This invention benefits both patients and surgical facilities, while reducing overall cost associated with operation and materials.

The invention may be used in conjunction with a surgical drain such as a Jackson-Pratt surgical drain having a soft plastic bulb with a venting port and a surgical drainage tube attached to it. The drainage tube is connected to a drain that is placed into the patient's surgical site through a small opening near the incision. When the bulb is compressed or squeezed with the venting port closed, a suction is created.

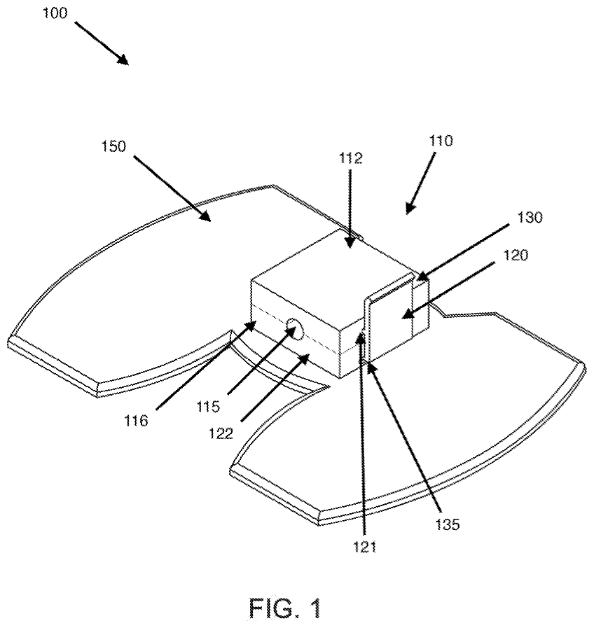

With reference now to FIG. 1, one exemplary embodiment of surgical drain immobilization system 100, according to the present invention is generally designated. Surgical drain immobilization system 100 may include a clasp such as clasp 110 that provides the means to secure a surgical drain by securing the drain tube inside clasp 110, a base such as base 150 that supports clasp 110, as well as provides leverage and means to immobilize clasp 110.

Surgical drain immobilization system 100 may be placed circumferentially around or approximate to a surgical drain site on the patient's body. Surgical drain immobilization system 100 may have a “V” notch design in base 150 that allows surgical drain immobilization system 100 to straddle the drain tube within clasp 110. Clasp 110 may be adhered to base 150 or in other non-limiting embodiments may be connected by fasteners, latches, hinges, welding techniques, or any other method known to those skilled in the art to connect clasp 110 to base 150 and immobilize clasp 110. Base 150 may be made of a pressure-sensitive vinyl material with a medical grade adhesive coated on at least a portion of the surface of base 150, opposite of clasp 110, for adhering to the patient's skin. The vinyl material allows for increased durability as it will not easily tear when stretched. In one or more non-limiting embodiments, a releasable liner may cover the adhesive so as to protect the adhesive from adhering to other objects before use.

Clasp 110 may include a top component such as top component 112 and a bottom component such as bottom component 116, top component 112 and bottom component 116 preferably rectangular in shape having one or more sidewalls, such as four sidewalls. Top component 112 and bottom component 116 may be connected using one or more hinges and pins. However, in other non-limiting embodiments snap-fit connections, fasteners, and other attachment mechanisms may be used. Hinge may be connected so that that top component 112 may be rotated at angle with respect to bottom component 116 and base 150 wherein when in a closed position top component 112 may be parallel to bottom component 116 and when in an open position top component 112 may be at an angle with respect to bottom component 116.

Top component 112 may have a bottom surface such as bottom surface 113 whilst bottom component may have a top surface such as top surface 117. Bottom surface 113 and top surface 117 may each have a semicircular recess over their common length, to provide a cylindrical profile aperture such as aperture 115 when clasp 110 is closed, for the surgical drainage tube to be coaxially placed with or positioned inside wherein aperture 115 surrounds surgical drainage tube. Aperture 115 may be of any shape and diameter that is larger or equal to a surgical drainage tube such that fluid is not prevented from flowing through the tube when clasp 110 is closed.

Bottom surface 113 and top surface 117 may include one or more studs or protrusions such as studs 122 which are fixed vertically downwards from bottom surface 113 and upwards from top surface 117. Studs 122 may be solid and integral with bottom surface 113 and top surface 117 and formed during molding of the tube or fastened by any means such as but not limited to fasteners, adhesives, latches, hinges, or welding techniques. The position of studs 122 on bottom surface 113 and top surface 117 are such that when a surgical drainage tube is positioned coaxially within aperture 115 of clasp 110, the surgical drainage tube engages tapered spike of bottom surface 113 and top surface 117. The position and size of studs 122 and aperture 115 created by bottom surface 113 and top surface 117 as illustrated are not intended to limit the invention to any particular combination of fluid volume, surgical drainage tube size, passageway and stud dimension, and a determination of satisfactory dimensions for these elements are well within the purview of one skilled in the art. The position in aperture 115 at which surgical drainage tube comes to rest may be determined by the diameter of aperture 115 and the dimensions of studs 122.

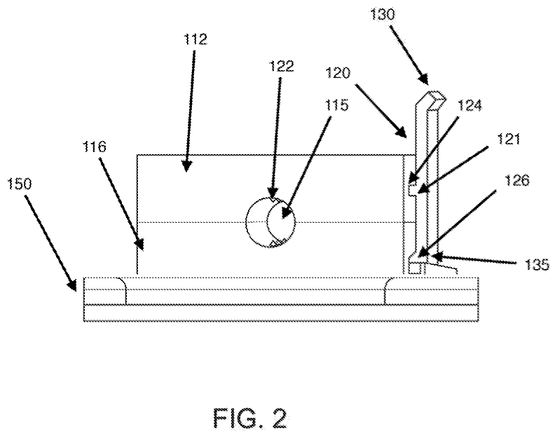

FIG. 2 shows a front view of the surgical drain immobilization system in the closed position. A recess such as recess 124 may be formed in a sidewall of top component 112 to connect to an actuator such as actuator 120. Actuator 120 may have a protrusion such as protrusion 121 coupled to recess 124 by several types of standard fastening devices or actuator 120 may be integral to top component 112 or otherwise be incorporated in the design of top component 112. The connection between protrusion 121 and recess 124 acts as a pivot point allowing actuator 120 to be rotated around the pivot point to lock and unlock clasp 110.

Actuator 120 may have a top end such as top end 130 and a bottom end such as bottom end 135. Top end 130 may have a general shape of a flexing curve of a thickened area that faces outward wherein top end acts as a lifting lever to open and close clasp 110. A recess such as recess 126 may be formed in bottom component 116 sized so that bottom end 135 of actuator 120 fits inside recess 126 wherein bottom end 135 comes in contact with the inside surface of recess 126. Bottom end 135 may mirror the top end 130 having a general shape of a flexing curve of a thickened area that faces inward wherein bottom end 135 acts as a tongue configured to be received in recess 126 of bottom component 116.

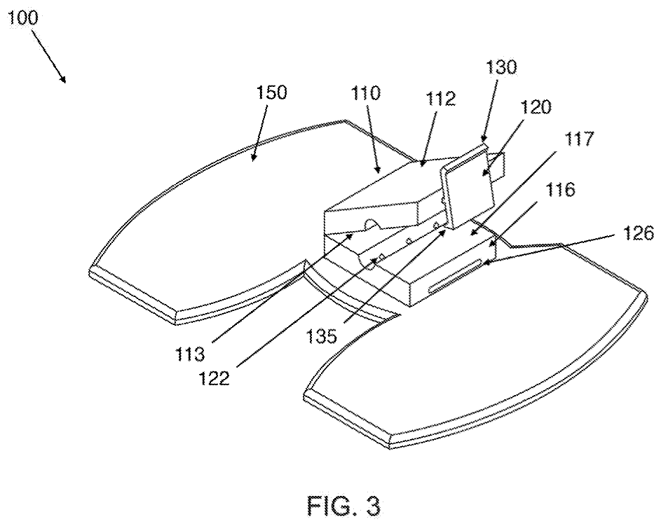

When clasp 110 is in the closed condition, top component 112 and bottom component 116 are secured together by the engaging contact of bottom end 135 to recess 126. To accomplish the opening operation of clasp 110, as illustrated in FIG. 3, the top of actuator 120 may be pushed or pulled toward top component 112 causing actuator 120 to pivot about a fulcrum in recess 124 of top component 112. Bottom end 135 in contact with the recess 126 moves outwardly away from recess 126 at a distance so that the edges of bottom end 135 and recess 126 may be parted and top component 112 and actuator 120 may be lifted away from bottom component 116. To accomplish the closing operation of clasp 110, the top of actuator 120 may be pushed or pulled toward top component 112 causing actuator 120 to pivot about the fulcrum in recess 124 of top component 112. Bottom end 135 may then be aligned with recess 126 wherein the tongue is no longer pushed or pulled toward top component 112 causing bottom end 135 to be received and secured in recess 126, thus locking top component 112 to bottom component 116.

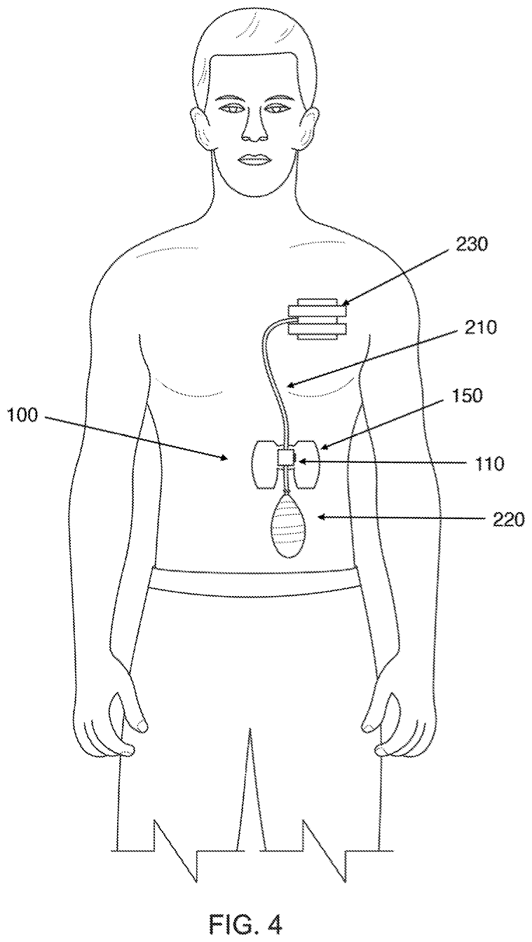

In use, as illustrated in FIG. 4, a surgical drain (not pictured) connected to a surgical drainage tube such as surgical drainage tube 210 is positioned within a patient's body at a wound site or other area such as wound site 230 wherein fluid has or will accumulate, during surgery or post-surgery, to allow the fluid to be drawn from wound site 230 into a bulb such as bulb 220. To initiate surgical drain immobilization system 100, an operator may adhere base 150 to the patient's skin at a predetermined distance to wound site or other area. The operator may then push or pull top end 130 of actuator 120 toward top component 112 wherein bottom end 135 of actuator 120 is removed from recess 126. Surgical drainage tube 210 is then positioned into aperture 115. Top end 130 of actuator 120 is then pushed or pulled toward top component 112 wherein top component 112 is placed parallel to bottom component 116 and the force to top end 130 of actuator 120 is removed causing bottom end 135 of actuator 120 to be received into recess 126, securing top component 112 to bottom component 116 and surgical drainage tube 210 inside aperture 115.

Once surgical drainage tube 210 is inside aperture 115, the surgical drain may be used similar to traditional methods wherein a venting port (not shown) of bulb 220 is opened, and the operator partially collapses bulb 220 by squeezing air from within bulb 220 out through the venting port. The operator then closes venting port, and releases the squeezing pressure on bulb 220, thereby generating a negative pressure within bulb 220. Negative pressure from bulb 220 is then transmitted through surgical drainage tube 210 to drain at or near wound site 230 such that drainage fluid is suctioned out of the patient and into bulb 210

After suction has been performed for the desired amount of time, the operator can open the venting port to allow the container to return to atmospheric pressure. The fluid contents of bulb 220 then can be poured out through the venting port into a recess for measurement or other further analysis. The operator then may squeeze bulb 220 and close venting port to reestablish suction. Once surgical drain immobilization system 100 is no longer needed, the operator may push or pull the top of actuator 120 toward top component 112 wherein bottom end 135 of actuator is removed from recess 126. Top component 112 is moved away from bottom component 116 and surgical drainage tube 210 is then removed from aperture 115. The operator then may remove base 150 from patient's skin.

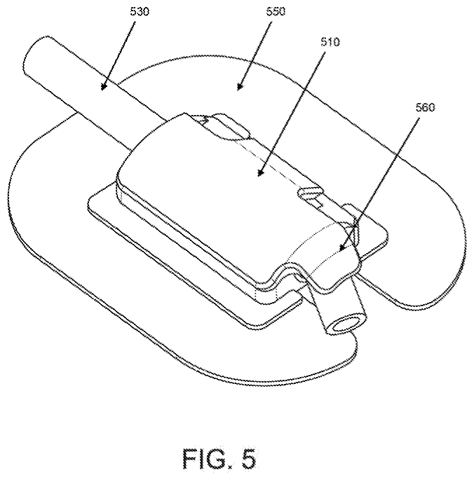

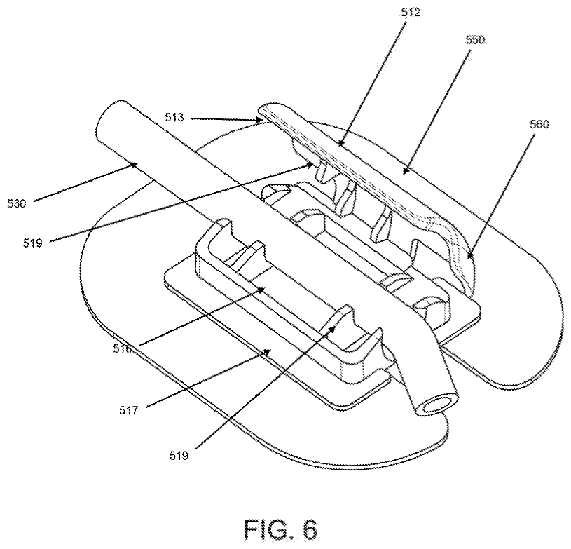

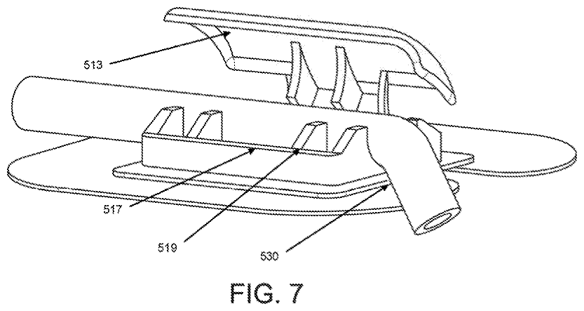

With reference now to FIG. 5-FIG. 7, a second exemplary embodiment of surgical drain immobilization system 500, according to the present invention is generally designated. Surgical drain immobilization system 500 may include a clasp such as clasp 510 that provides the means to secure a surgical drain by securing the drain tube inside clasp 110, a base such as base 550 that supports clasp 550, as well as provides leverage and means to immobilize clasp 510.

Clasp 510 may be adhered to base 550 or in other non-limiting embodiments may be connected by fasteners, latches, hinges, welding techniques, or any other method known to those skilled in the art to connect clasp 510 to base 550 and immobilize clasp 510. Base 550 may be made of a pressure-sensitive vinyl material with a medical grade adhesive coated on at least a portion of the surface of base 550, opposite of clasp 510, for adhering to the patient's skin. The vinyl material allows for increased durability as it will not easily tear when stretched. In one or more non-limiting embodiments, a releasable liner may cover the adhesive so as to protect the adhesive from adhering to other objects before use.

Clasp 510 may include a top component such as top component 512 and a bottom component such as bottom component 516, top component 512 and bottom component 516 preferably rectangular in shape having one or more sidewalls, wherein top component 512 has a convex surface allowing for a surgical drainage tube such as surgical drainage tube 530 to be placed into clasp 510. Surgical drainage tube 530 may have predominantly flat and then curve at angle at an end for securing to an attached bulb. Top component 512 may have a tongue such as tongue 560 that extrudes from the body of top component 512 at a downward angle to secure and protect the end of surgical drainage tube 530 that is at a downward angle with respect to the rest of surgical drainage tube 530.

Top component 512 and bottom component 516 may be connected using one or more hinges and pins. However, in other non-limiting embodiments snap-fit connections, fasteners, and other attachment mechanisms may be used. Hinge may be connected so that that top component 512 may be rotated at angle with respect to bottom component 516 and base 550 wherein when in a closed position top component 512 may be parallel to bottom component 516 and when in an open position top component 512 may be at an angle with respect to bottom component 516. Top component 512 and bottom component 516 may be preferably connected when closed snap shut type mechanism wherein top component 512 may be snap shut with respect to bottom component 516 and then pulled open by applying upward force on a lip of top component 512.

Top component 512 may have a bottom surface such as bottom surface 513 whilst bottom component 516 may have a top surface such as top surface 517. Bottom surface 513 and top surface 517 may each have a protrusions such as protrusions 519 with a semicircular recess over their common length, to provide a cylindrical profile when clasp 510 is closed, for surgical drainage tube 530 to be coaxially placed within the cylindrical profile to surround and secure surgical drainage tube. The cylindrical profile may be of any shape and diameter that is larger or equal to a surgical drainage tube 530 such that fluid is not prevented from flowing through the tube when clasp 510 is closed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A drain immobilization device, comprising:
a clasp configured to secure a surgical drainage tube, the clasp connected to a base, the base configured to attach to a patient's skin.

2. The drain immobilization device of claim 1, wherein adhesive is coated on at least a portion of a surface of the base.

3. The drain immobilization device of claim 2, the base comprised of a vinyl material.

4. The drain immobilization device of claim 3, the clasp comprised of a first component and second component, the first component pivotably rotatable to the second component.

5. The drain immobilization device of claim 4, the first component and the second component each having a recess, the recess of the first and the second component integrally forming an aperture when the first component and the second component are parallel to one another, the aperture configured to receive the surgical drainage tube.

6. The drain immobilization device of claim 5, the recess of the first and the second component having one or more studs along the length of the recess of the first and the second component.

7. The drain immobilization device of claim 6 further comprising: an actuator configured to lock and unlock the clasp.

8. The drain immobilization device of claim 7, the actuator having a bottom end configured to be received into a recess in the sidewall of the bottom component.

9. The drain immobilization device of claim 8, the actuator having a top end configured to be moved toward a sidewall of the top component such that when top end moves toward the sidewall of the top component the bottom end is moved away from the recess in the sidewall of the bottom component.

10. The drain immobilization device of claim 9, the top end and the bottom end in the shape of a flexing curve, the top end facing outward from the sidewall of the top component and the bottom end facing inward toward the sidewall of the top component.

11. The drain immobilization device of claim 10, a part of the actuator configured to connect to a recess in the sidewall of the top component, the connection configured to act as a pivot point for the actuator to rotate around.

12. The drain immobilization device of claim 11, the clasp connected to the base by an adhesive.

13. A drain immobilization device, comprising:
a clasp configured to secure a surgical drainage tube, the clasp comprised of a first component and second component, the first component pivotably rotatable to the second component.

14. The drain immobilization device of claim 13, the first component and the second component each having a recess, the recess of the first and the second component integrally forming an aperture when the first component and the second component are parallel to one another, the aperture configured to receive the surgical drainage tube, an actuator configured to lock and unlock the clasp, the actuator connected to the clasp such that the connection acts as a pivot point for the actuator, the actuator having a second end configured to be received into a securing recess in the clasp wherein when the second end is received into the securing recess the surgical drainage tube is secured in the aperture, the actuator having a first end configured to be moved toward the clasp such that when first end moves toward the clasp the second end is moved away from the securing recess in the clasp, the first end and the second end of the actuator are in the shape of a flexing curve, the first end facing outward from the sidewall of the first component and the second end facing inward toward the sidewall of the first component.

15. The drain immobilization device of claim 14, the first component and the second component each having one or more protrusions having a recess, the protrusions integrally forming an aperture when the first component and the second component are parallel to one another.

16. The drain immobilization device of claim 15, the recesses of the one or more protrusions semicircular in shape.

17. The drain immobilization device of claim 16, the first component having a tongue extruding from a body of the first component, the tongue at an angle with respect to the body.

18. The drain immobilization device of claim 17, the clasp connected to a base, the base configured to be attached to a patient's skin, wherein adhesive is coated on at least a portion of a surface of the base.

19. The drain immobilization device of claim 18, the base comprised of a vinyl material.

20. The drain immobilization device of claim 19, wherein adhesive is coated on at least a portion of a surface of the base.
